(19) United States
(12) Patent Application Publication
(10) Pub. No.: US 2015/0040949 A1
(43) Pub. Date: Feb. 12, 2015

(54) ARRANGEMENT AND METHOD FOR PREPARING A STENT FOR IMPLANTATION

(71) Applicants: Arik Zucker, Zurich (CH); Stefano Buzzi, Zurich (CH); Armin W. Mäder, Richterswil (CH); Roman Schneeberger, Brittnau (CH); Vincent Milleret, Zurich (CH)

(72) Inventors: Arik Zucker, Zurich (CH); Stefano Buzzi, Zurich (CH); Armin W. Mäder, Richterswil (CH); Roman Schneeberger, Brittnau (CH); Vincent Milleret, Zurich (CH)

(21) Appl. No.: 14/371,711

(22) PCT Filed: Dec. 20, 2012
(86) PCT No.: PCT/EP2012/076406

§ 371 Date: Jul. 10, 2014

(30) Foreign Application Priority Data
Jan. 11, 2012 (CH) 48/12

(51) Int. Cl. B08B 3/04; A61F 2/95; A61F 2/82

(52) U.S. Cl. CPC B08B 3/04; A61F 2/82; A61F 2/95
USPC 134/22.11; 134/166 C; 134/88; 53/523; 53/436; 206/438

(57) ABSTRACT

A method and an arrangement are disclosed for providing an implant for implantation in a body lumen, that include at least one processing unit (1) for processing the implant (7; 103), a sheathing (2) with a front opening (3) and a rear opening (4), which is at least partially disposed at or in the processing unit (1), and at least one power unit (5). The implant (7; 103) can be stored inside the sheathing (2) between the openings (3, 4). The power unit (5) serves for generation of a current (P) of a defined medium through the sheathing (2), which is connected at least to one opening (3; 4) of the sheathing (2), whereby the current (P) flows inside the sheathing (2) with the stent (7; 103).

The present invention relates to the preparation of an implant, in particular a stent, for implantation and/or for storing of an implant prior to implantation, an arrangement for handling and/or making available an implant as well as a packaging for an implant.

Small implants, such as stents, are in widespread use in a multiplicity of medical applications. Stents are used, for example, for treatment of lesions in blood vessels or in treatment of aneurysms or for vessel dilation in the field of percutaneously transluminal angioplasty, among other things also for cardiovascular intervention.

A distinction is made substantially between balloon-expanding or self-expanding stents. Balloon-expanding stents are placed prior to their placement on a non-expanding balloon. For this purpose, for example, the stent is compressed to a smaller diameter over the non-expanding balloon and together with the balloon are introduced into the body by means of a transport device. At the treatment point, e.g. in the case of a lesion or a vessel valve, the balloon is expanded so that it expands the stent. Subsequently the balloon can be removed from the body. Self-expanding stents can consist of a metal with memory effect. For introduction into a body lumen, they can be compressed, toward their expansion power and can be inserted into a tube catheter. At the place of treatment, they are released from the catheter (e.g. pushed out of the tube catheter) and spring back into their expanded status.

Furthermore a distinction is made between bare metal stents and stents with coatings. Soehnlein et al. (Neutrophil-Derived Cathelicidin Protects from Neointimal Hyperplasia; Sci Trans! Med 3, 2011) shows e.g. a stent having a protein coating with the protein cathelicidin (LL37). The incorporation behavior of the coated stent was tested on animals. The trials showed a minimal restenosis and it is assumed that Cathelicidin promotes and regulates the reendothelialization, and thereby protects against a neointimale hyperplasia after the stent implantation. The use of coatings on stents however in general increases, for its part, diverse drawbacks, e.g. the surface friction of the stent can be increased, the coating can be damaged during the expansion of the stent (tearing, peeling, etc.) or generate negative side effects with the blood circulation.

Common to all stents is that, for insertion into a body lumen, they must have a diameter more minimal than in the exercise of their function in the body. As a rule, the stents are prefitted on a catheter by a manufacturer and packaged. As a rule, however, it is also possible to compress a stent only shortly before insertion of the stent into the body lumen, i.e. crimping. With conventional methods for providing a stent for implantation, the stent is compressed, usually in expanded or semi-expanded state, on the catheter to a smaller diameter suitable for introduction into the body of a patient. This procedure takes place usually under so-called sterile conditions. A sterile room is a room in which the concentration of airstored particles brought into the room or arising there is kept as minimal as necessary. The particle concentration in the sterile rooms is constantly monitored. Sterile rooms are arranged in different classes, so-called ISO classes, thus e.g. in a sterile room of the class ISO-7 the number of particles that are 0.5 Micrometers in size must not measure more than 352 000 per cubic meter. For 1.0 micrometer the upper limit is 83 200 and for 5.0 micrometers 2930 particles per cubic meter (according to DIN EN ISO 14644). Particles smaller than 0.5 micrometers are not taken into consideration in this class. For medical implants, such as e.g. stents, such an ISO-7 sterile room is used.

A sterile room for manufacture and preparation of stents on catheters is therefore not 100% particle-free. Moreover many production steps are carried out by hand precisely for stents and catheters. Since, as a rule, the human being is the largest source of particles and other soiling, suitable work clothing helps to keep the specified sterile class.

The stents implanted in blood vessels bring with them certain risks for the patient. Among other things reactions of inflammation and/or thrombosis on the structures of the stents can lead to a renewed stenosis in the blood vessels. Complications of this kind are brought about, among other things, by soiling of the stents, the insertion device or other elements that come into contact with the stent during introduction of the stent into the body lumen. The stent and the transport device should therefore be generally kept free of any contamination, i.e. for example no dust, no fibers or particles.

With conventional methods for providing a stent for implantation, e.g. also after a surface treatment and cleaning of the stent, such soiling can arise e.g. during transfer of the stent, during crimping or insertion on or in a catheter. For example, natural contamination, e.g. of work gloves or diverse particles from the atmosphere, through work carried out in the sterile room can remain on the stent.

Through optical checks after installation of the stent on the catheter, any particles can be found and if necessary removed by means of a clean gas flowing in out of a nozzle.

Particles which are located, however, between the mounted stent and the catheter, cannot be found depending upon conditions. Thus with a balloon-expanding stent, particles can be located, among other things, between the mounted stent and balloon and with a self-expanding stent between installed stent and inner tube of the tube catheter. Minimal particles, which cannot necessarily be optically found, and also do not come under the applicable ISO norm, likewise remain on the surfaces of stent and catheter. Moreover these optical checks are often carried out manually, which can mean an additional source of error.

Known from the state of the art, certain types of imcleanliness are to be avoided on an implant or a transport device. Shown in WO 2006/086709 is, for example, a device for compressing nitinol stents. The expanded stent is cooled from a first temperature down to a second temperature and then compressed. The problem thereby arises that, during cooling, gaseous materials, such as e.g. water vapor or other sublimates found in the air on the surface of the stent can condense and thus freeze, and thereby impede the compression and/or damage the stent surface, which, among other things, can be the case with coatings. In order to prevent this, the stent and the crimping device are provided in a closed-off chamber and any gaseous material that can condense during the cooling process is removed as much as possible, such as e.g. water vapor and carbon dioxide. Only then is the stent cooled to the compression temperature. For removing the gaseous materials, the chamber is supplied with a gas, such as e.g. nitrogen, so that the stent and the crimping device are situated in a nitrogen environment. This is however not sufficient since, still smaller contamination particles can still be present in the micrometer range, which are present e.g. already before the exchange of gases on the stent surface or on the balloon surface, but however do not influence the crimping procedure.

Shown in the US 2006/0183383 is a crimping device for compressing a stent, which is used as a protective layer between stent and crimp elements of the device in order to protect the stent during compression. The protection is brought into shape of a rollable material within the crimping device and comes to be between the stent and the crimping device. The protective layer, at its ends, is in each case to be rolled between the stent and crimp elements, between the area tensioned between the rollers there exists a longitudinal opening, which for example occurs through the removal of a crimping element by means of a pressurized stream of a gas between the elements into the inside of the crimping device. As soon as the crimping device for compression of the stent is closed, the gaseous stream must be adjusted and the gas can escape through the fore and rear opening of the crimping device, so that the stent is released into the atmosphere. After the compression, the stent will be taken away out of the crimping device, and through transfer be again subjected to a contamination.

Furthermore shown in US 2003/0187493 is a packaging sheath for a compressed stent and a catheter, which is shown mounted on the stent. The sheath does not come into contact with the surface of the stent, or respectively the coating in order not to damage this (for example when it concerns a coating) or to soil this, for example when the stent is brought in and put on a transportation device for implantation. Impurities in the form of micro particles (or smaller) which were already present on the stent before packaging thus stay on the stent.

Further devices for compression of a stent or its preservation are known from U.S. Pat. No. 6,968,607, US 2008/0072653, WO 2004/066876 or EP 0910425, for example.

In the devices and methods for preparation and processing of small implants, such as stents, these kept is kept within the device or during the method in a pure environment and soiling is avoided as much as possible. After the processing, e.g. during transfer from one working unit to the next, or with a simple check of an implant, however, impurities again can accumulate again on the surface of the implant. In the case of a stent, such lack of cleanliness can occur between the stent and its transport catheter, and be brought into a body lumen along with the stent, where they can lead to problems such as has been described in the foregoing.

It is a object of the present invention to create an arrangement and a method for providing an implant for implantation that reduces impurities on implants during the preparation for implantation and with the packaging, depending upon a cleaning process in order to prevent repeated impurities, to preserve the quality of a surface of an implant and which is easy in the implementation and handling. Furthermore it is an object of the invention, to create a packaging for an implant in order to make possible a coating free of recontamination and uncomplicated in its application.

This object is achieved by the invention through an arrangement for preparation of an implant for implantation as well as a packaging according to claims 1, 14 and 25. Advantageous arrangement and different embodiments are described in the dependent claims.

The present invention is suitable for implants in general, in particular for small implants such as vascular prostheses, for example stents. Stents have substantially a proximal end and a distal end, whereby a stent lumen of compressible diameter extends between the ends, which, as a rule, is composed of individual crosspieces. As described at the beginning, stents are disposed on a transport device, e.g. a catheter, in order to be transported inside a body lumen. The present invention is suitable to prevent impurities between stent surface and catheter surface, e.g. in the form of micro particles, as described in the foregoing.

With a method for providing an implant, in particular a stent, for implantation in a body lumen, the implant is inserted within a sheath of a processing unit a packaging, and preferably removed. Thereby the implant is stored between a fore and a rear opening of the sheath, and a current of a defined medium is applied through the sheath. The current flows around the implant in the sheath and the processing unit takes effect on the implant outside the sheath.

Used thereby, according to the invention, is an arrangement for providing an implant, in particular a stent, for implantation in a body lumen, which at least comprises a sheath with a front opening and a rear opening, and at least one current unit for generating a current of a defined medium through the sheath, the current flowing around the implant within the sheath from all sides. The flowing unit can be connected immediately to the sheath.

Alternatively, a packaging, for example, can be placed between the sheath and the implant, so that an implant can be transferred out of the sheath in the packaging without an interruption of the current around the implant. The sheath is disposed at least partially at or in the processing unit or can be disposed therein or thereon, whereby the implant is stored inside the sheath between the openings thereof. The current unit for generating a current through the sheath is at least connected to an opening of the sheath. The openings thereby form a flowing-in and flowing-out opening. The sheath can have a conventional attachment system for attachment to the current unit, such as, for example, a plug-in or screw connection. At the same time, a stent has preferably current flowing around it on all sides, i.e. on its outer and inner circumferential surface and also between individual bridges of the stent. The invention is described in the following, by way of example, in the form of an implant as a stent. The invention can, however, be implemented also advantageously for other implants such as e.g. grafts or teeth implants.

Experimentally it could be shown that there was reduced restinose according to the invention for implantation of prepared stents. This can be attributed to the fact that stents prepared and stored have, compared to conventionally prepared and stored stents, a higher degree of cleanliness at the point in time of the implantation, i.e. the contamination is clearly reduced. With the aid of the invention, a consistent degree of cleanliness can be maintained or achieved over the entire stent surface. There remain no particles such as germ cells for an irregular growing together. In particular with hydrophilic stents it is possible with the procedure and the arrangement according to the invention, to keep the high degree of cleanliness of the hydrophilic surface for insertion of the stent into the body lumen.

In a preferred embodiment, it is at the same time, a transport device is disposed for insertion of the stent into the body lumen, such as, for example, a catheter, inside the sheath, between the orifices thereof, and a defined medium flows around it, such as carried out more precisely in the following.

With the method and the arrangement according to the invention, the stent can be subject to a current of a defined medium during the processing, storage and until introduction in a body lumen, whereby it preferably remains with every step of treatment within the sheath. Thus, for example, also during the introduction into a processing unit and during the processing a current prevails, the contamination particles are transported out of the sheath. The storage of impurities is thereby prevented.

The stent can, e.g. be disposed within the sheath and be purified therein by means of a cleaning unit. For cleaning, an ultrasound unit is used, for example, which takes effect on the stent from the outside of the sheath in known way. At the same time, the stent can be rinsed inside the sheath with a solution. A base solution, e.g. with 3% Deconex in distilled water can be used. It is also possible, to use an acidic solution, such as e.g. concentrated sulfuric acid (e.g. 96%). The material of the sheath is determined based on the type of solution. In the case of sulphuric acid and/or Deconex solution a Teflon tube (PTFE) can be used. The solution can thus be a defined medium in the sense of the invention. Fundamentally also other conventional methods are also applicable. Usually the sheathing can consist of plastic, for example PA and PE. Good results have been achieved with PTFE and PFA.

The stent thus remains and after the cleaning inside the sheath and the surrounding current of the defined medium rinses the stent. In this protected atmosphere, the stent can be fed into a processing unit, without there being in the meantime a contaminated atmosphere.

Thereby the defined medium can also be changed for the stent during individual steps of the preparation method. For example, before and/or after a current consisting of a solution for purification another, e.g. nitrogen current, can be introduced through the sheath, so that the stent can be rinsed without an interruption of different currents following subsequently. For example, before and/or after a stream consisting of a solution for purification of other e.g. nitrogen can be conducted through the sheath, so that the stent is rinsed. To this end, the front opening of the sheath can be provided with e.g. a plurality of inlets, or openings, through which the different media are able to be introduced into the sheath. A Y-element with two inlets on the front opening of the sheath can be attached and at every access a current unit for a particular medium can be connected. Fundamentally also three or more inlets for three or more current units can be provided. The different media can also be mixed inside the sheath. The mixing ratios can also be determined thereby, e.g. through adjustment of the current units.

In an embodiment, the sheath is of tubular form and is designed flexible and bendable. The sheath can extend at two ends of the implant, or respectively of the stent, far beyond this, so that the sheath can extend beyond a multiplicity of processing units. Owing to flexibility of the sheath, effects outside the sheath can affect the stent and the stent can e.g. be compressed. The surface of the stent thus remains however protected. Provided can be holding means for positioning of the stent inside the sheath or for grasping the stent from outside. This can e.g. provided in form of one or more taperings of the diameter of sheath through compression from outside, for example by clamping or manually. The stent can e.g. be stored detached at the taperings. Thus the stent can be kept inside the sheathing loosely with minimal play, so that it remains accessible for the current of the defined medium. The stent can also be kept detachably outside the sheath on its circumferential surface. Substantially the holding means are designed in such a way that the stent despite an attachment there can be flow through the holding means as much as possible on all sides.

At the same time, with the stent, a transport device can be provided for introduction of the implant into a body lumen together with the implant. In the case of balloon catheter as transport device, the procedure can take place as follows. The stent is introduced into the sheath and the medium flows around it. Subsequently the balloon catheter is brought in and likewise the medium flows around it. After a while, while the medium flows around stent and balloon, and there are thus no particles anymore on their surfaces, the balloon can be pushed again inside the body lumen. The balloon can then be opened somewhat, i.e. inflated, so that the stent through adhesive friction remains on the balloon. Stent and balloon are then pushed as unit into the crimping device and reduced in diameter.

Used as sheath can be e.g. a hose of PTFE, PFA, PE or PA. The tube is cleaned before its use as sheath for the stent. It is advantageous if the sheath is transparent in order for a visual control to be carried out around the stent in the sheath. Preferably the sheath has a wall thickness of 0.01 mm to 0.09 mm auf, in particular 0.02 mm to 0.06 mm. In this area a sheath is sufficiently flexible and bendable in order not to impede the required processing steps, but sufficiently robust, in order not to tear during a processing of the stent. In particular suitable is, for example, a PTFE tube with a wall thickness of 0.04 mm.

The processing unit can be formed by a crimping unit that compresses a stent, in order to bring it onto a balloon catheter or to bring it into a catheter for self-expanding stents. The crimping unit has elements which are disposed around an axis and are movable at least partially with respect to one another radially in relation to the axis, whereby the free space which encloses the elements becomes narrower. The elements of the crimping unit comprise the stent positioned in the sheath, and are movable radially from an expanded position, in which the stent is uncrimped or respectively uncompressed, into a closed position in which the stent is compressed in diameter. In the expanded position of the elements the free space which encloses them has at least such a diameter that the stent in expanded state is able to be accommodated in this space. In the closed position of the elements, the elements move inwardly in so far that the free space is reduced to a diameter corresponding to the diameter which is foreseen for a compressed or respectively stent.

The elements of the crimping unit can be designed as pivotable jaws or as segments which are disposed in a circle around an axis and are movable in direction of the axis. Crimping units are known from the state of the art.

A stent can be inserted within its sheath, for example in radial or axial direction into a crimping unit. Preferably the sheath extends through the crimping unit and comes out on the other side of the crimping unit. The sheath thus protrudes out of the crimping unit. Thus the stream unit can remain attached to the sheath, and the stent can through adjustment of the sheath be moved inside the crimping unit in order to bring it e.g. into a desired position with respect to the unit. Thereby the crimping elements come to be situated outside the sheath above the stent, and can have an effect thereon without the stream of the defined medium having to be interrupted.

In an embodiment of the arrangement according to the present invention, a multiplicity of processing units is provided in sequence, whereby the sheathing extends through the multiplicity of processing units. The implant or respectively the stent can thus be moved within the sheath from a processing unit to one of the next. Alternatively, the sheath can be moved by the processing unit 6s until a stent substantially stationary inside the sheath is moved from one processing unit to the following processing unit or also in a packaging. During this transfer, the stream of the defined medium can flow around the stent continuously, so that the deposits of impurities are avoided.

Fundamentally however it is also possible for the sheath to be removable from a processing unit so that an implant located in a sheath, or respectively a stent, is able to be transferred from one processing unit to a subsequent processing unit without there being any imcleanliness.

As processing unit, a crimping unit, as described above, a cleaning unit, a unit for tempering (increasing or lowering the temperature) of the implant or respectively a packaging unit, or respectively a packaging can be provided in the arrangement, for example. Depending upon requirements with respect to be implant, other processing units can also be provided.

The flow unit for generation of a current of a defined medium through the sheath can in a simple embodiment and by means of a container in which the medium is located and by means of generated pressure/overpressure or gravitation flows into the sheath through an opening. A gas, nitrogen or a liquid can be used as the defined medium, for example. The composition of the medium can be selected with respect to the requirements of the implant. Preferably an inert medium is used, which respect to the surface of a stent is not reactive inside the sheath. For example sterile salt solution in physiological concentration of 0.9% NaCl can be used for this purpose. This has the advantage that the stent is kept in a physiologically suitable environment. Water may also be used for injection. Furthermore defined substances can be admixed with the medium, for example through the mixture inside the current unit or inside the sheath, such as within the flow unit or within the sheath, as described by way of example in the foregoing. Through the defined substances the surface of a stent can be preserved, for example. Also substances can be used in order to influence the surface characteristics of a stent, for example oxidation means (e.g. hydrogen peroxide, H₂O₂, or also nitric acid) in order to oxidize the stent surface. The flow unit can have pressure regulation for regulation of the pressure inside the sheath. Thus it can be ensured that a consistent flow flows through the sheath even if the diameter of the sheath is changed during the processing. With such a closure of the sheath, the pressure regulation can also shut off the stream. The strength of the stream can also be varied, e.g. in harmony with various e.g. processing steps prior to a packaging of the stent.

According to the invention, the implant is preferably surrounded by flow while the implant is cleaned, processed, a packaging is added and/or transferred between unit. Preferably the flow unit generates inside the arrangement a retrograde flow of the medium with respect to the transport device of an implant within the arrangement. The current thus runs within an arrangement from one processing unit in direction of a cleaning unit. The cleaning unit is thus provided in flow direction according following a processing unit. Preferably the flow also flows through a packaging unit, or respectively in or through the packaging itself, and from there in direction of the cleaning unit.

The packaging is preferably disposed at the front, i.e. as first unit after the flow opening for the define medium, for example. By means of an opening on the flow unit and with a facing opening, the packaging unit can thereby be attached to the packaging unit in flow direction. The defined medium thereby flows around the packaging. Thus the packaging is also purified with respect to any possible impurities/contamination particles, and the surface remains clean until insertion of the implant or fitting of the implant onto a transport device. The flow direction runs, as a rule, in longitudinal direction of the implant, in particular of the stent, and thus in longitudinal direction of the processing axis, such as, for example, the longitudinal direction of the crimping device. Insofar as a transport device is provided for the stent within the sheath, this is also aligned in longitudinal direction. The implant is thereby surrounded at all times with fresh, uncontaminated medium during the preparation of the implant. Thereby there is a continuous stream inside the sheath. The stream speed can be adjusted to be constant or variable. Thus, in the course of preparation of the stent for implantation, various conditions can be considered. For example it is advantageous during the transfer between various processing units or to a packaging, to achieve a heightened flow speed in order to prevent any particles which could lead to contamination by means of the flow from the stent. Fundamentally it is conceivable that the flow direction of the flow can run in reverse direction or vice versa. Furthermore it is conceivable to have various media or respectively substances flow in an alternating manner and/or from different directions through the sheath. This can take place, for example, by means of the same flow unit or through further flow units on the same end or opposite ends of a sheath.

It is also possible to change the temperature of the flow during the processing of the stent. For processing of the stent, the change in temperature can also thereby be used. In particular in the processing of self-expanding stents, there is a lowering of the temperature of the medium for processing of the stent. The stent can thereby be cooled e.g. with austenite formation temperature (AF temperature) of the stent material in order to eliminate the memory effect with respect to the materials. For nitrinol stents, this temperature is, for example, adjusted to below room temperature. Depending upon the cooling of such a stent, a tube catheter can be disposed above the stent in a further processing step, while the flow flows around the compressed stent.

Together with the implant, a transport device, such as an insertion catheter or a transport catheter for balloon-expanding or self-expanding stents, can also be disposed within the sheath, and thus be flowed around by the flow. The transport device is thereby likewise protected from impurities until this is used for insertion of the stent in a body lumen.

According to the invention, it is especially advantageous if the stent or is able to be put on or in the transport device while it is flowed around by a defined medium, in particular along the longitudinal axis of the stent. Contamination particles that are found between stent and transport device can thus be removed from the flow and e.g. with a crimping method and not be clamped between transport device and stent, where they are removed only with the release of the stent inside the body lumen. It is thereby advantageous that the flow can take place continuously during the entire processing. Preferably at least before and/or during the positioning of the stent, on or in the transport device, a current must be generated which flows around the stent and keeps away contamination.

Following a processing procedure, the implant or respectively the implant together with the transport device can be guided into a packing unit or directly into a packing. Preferably at least a portion of the sheath is thereby disposed inside the packaging. The implant thus remains also inside the packaging in the protected sheath. The packaging is closed via the sheath so that the openings of the sheath are closed. Until closing of the packaging, the flow can flow through the sheath. Protruding parts of the sheath can be lengthened. Such a processed and packaged stent is thereby free of contaminated particles to a far-reaching extent, and can, after removal from the packaging, can be directly inserted into a body lumen, without further processing steps being necessary, such as cleaning or compression.

According to the invention, a packaging of an implant for implantation in a body lumen is thus provided having the one tubular flexible sheath with a front opening and a rear opening between which the implant is situated, whereby the sheath is provided inside the packaging and the opening are closed by the packaging or parts of the packaging. At least at one end of the openings of the sheath there can be a connection for a flow unit for generation of a flow of a defined medium through the sheathing.

Alternatively, the packaging can have two openings, which enclose there-between a packaging space, whereby the sheath can be connected to a first opening and the flow unit to a second opening of the packaging. The flow of the defined medium thus flows from the flow unit through the packaging and further from the packaging through the sheath. At all times during the processing and the packaging the stent is rinsed by the flow and is thereby protected while it is transferred from the sheath into the packaging. Then the openings of the packaging are closed so that the stent and also the transport device belonging thereto are able to be kept in the protected environment.

The invention relates furthermore to an implant in the form of a stent, which is prepared according to the method described above for implantation. Through the preparation method a highly pure stent can be prepared having a uniform or periodically regular surface moistening characteristic. Compared with conventionally prepared stents, a uniform stent surface prepared according to the invention improves the behavior of the stent during growing in and acts against a restenosis. An experimental verification therefor has been able to be provided with in vivo studies, in which stents according to the invention were placed inside pigs and the growing in was monitored as will be explained more precisely in the following. As a surprising result it could be shown that the growing in characteristic of stents is essentially more dependent on the cleanliness of a stents than previously assumed. As the measurement results show, surprisingly good results were obtained with stent treatments using bare metal stents. Previously it was assumed that results of this kind could only be achieved with special preparations of the surface, e.g. through coating, for instance a medical coating, of the stent. The experiments show however that the success of a treatment of a body lumen with a stent depends primarily on the cleanliness of the stent.

In an advantageous embodiment, the entire surface of the stent has a uniform cleanliness having fewer particles per surface unit than corresponding to a conventionally used cleanroom classification (e.g. 7) of the ISO (International Standard Organisation). That means that, with the proposed method, a higher cleanliness of the stent surface can be achieved than is possible in clean rooms of the highest usually used cleanliness classes without such a clean room or even clean room of a lower level being necessary. In particular the stent can also have fewer carbon molecules and particles of a size less than 0.05 micrometers than corresponding to a cleanroom classification 1 of the ISO (International Standard Organisation).

The surface characteristic of the stent can be verified advantageously through a method and a device according to the parallel of the applicant having the title “Method und Vorrichtung zur Bestimmung einer Oberflächencharakteristik an Stents und Stent mit definierter Oberflächencharakteristik” (application number CH 00049/12). The disclosure of this application in relation the surface characteristics of an inventive stent and the determination of the surface composition is incorporated in full.

Preferred embodiments of the invention will be presented in the following with reference to the drawings which serve merely for explanation and are not to be interpreted in a limiting way. Features of the invention disclosed from the drawings should be viewed individually and in combination as belonging to the disclosure of the invention. Shown in the drawings are:

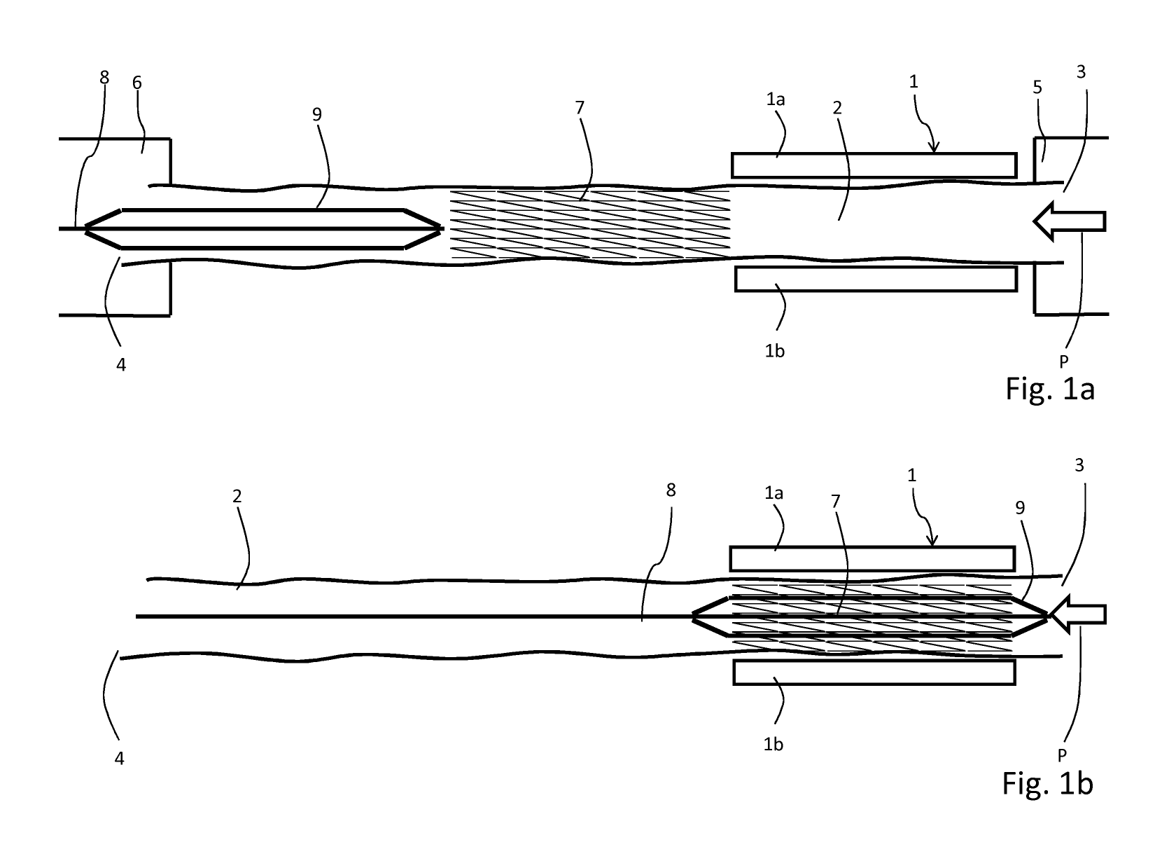

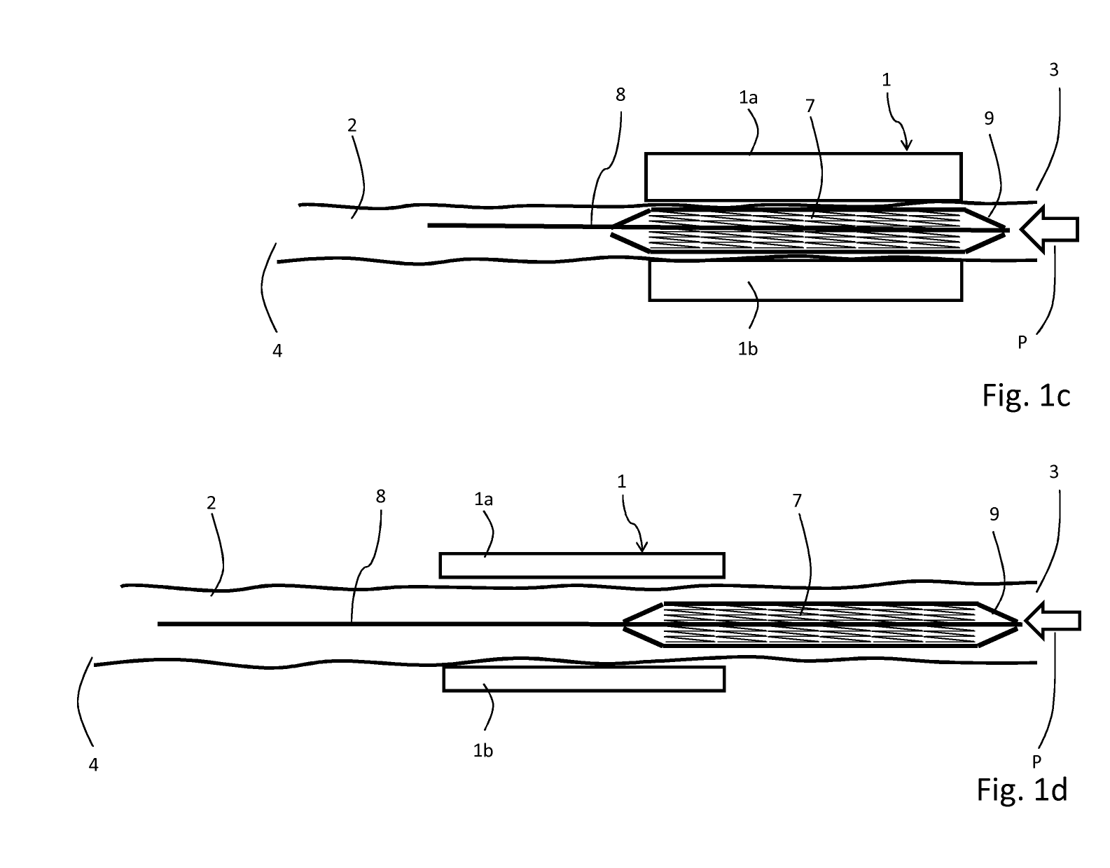

FIGS. 1a to 1d: a first variant of the method according to the present invention with a first arrangement according to the invention for a balloon-expanding stent.

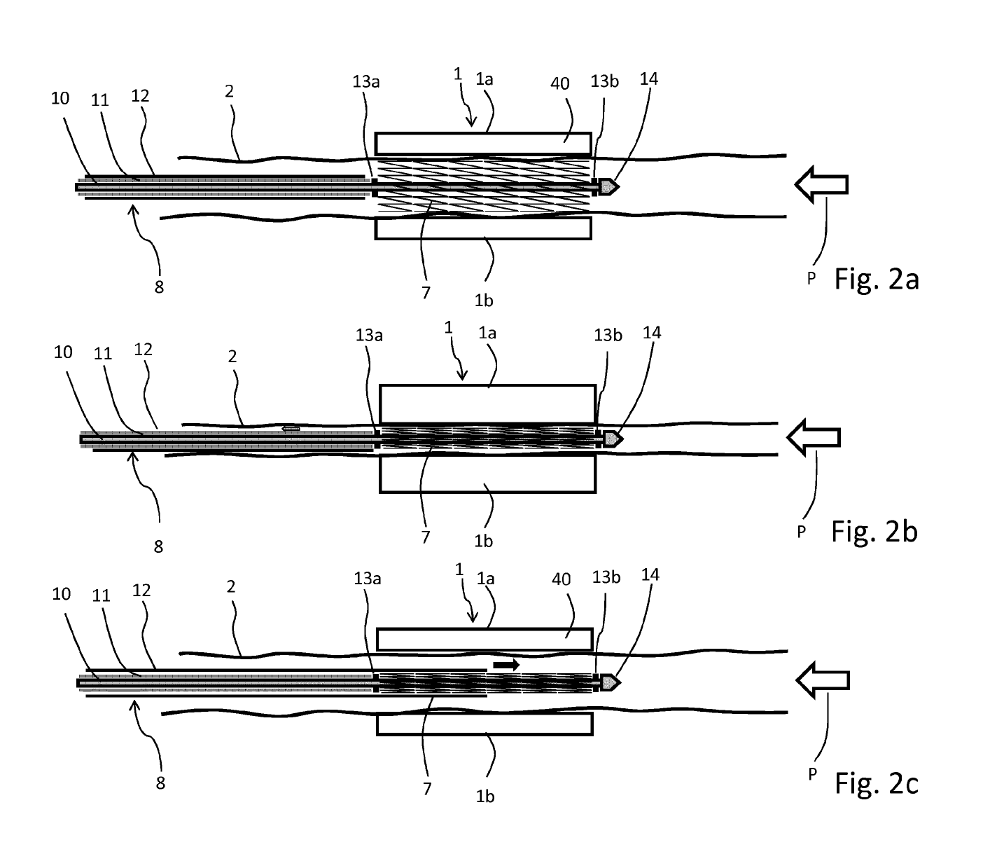

FIGS. 2a to 2c: a second variant of a method according the present invention for a self-expanding stent.

FIG. 3: a third variant of the method according to the invention with a second arrangement according to the invention.

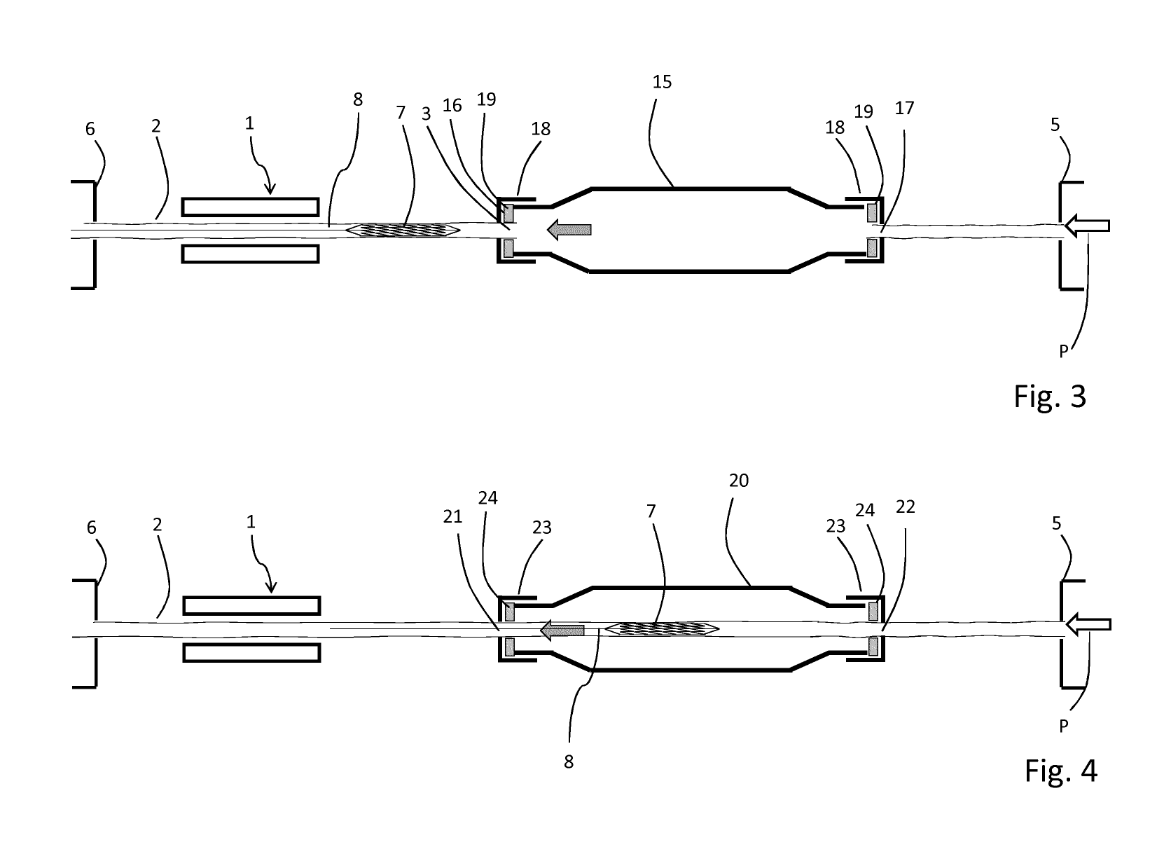

FIG. 4: a packaging with a sheath according to the invention.

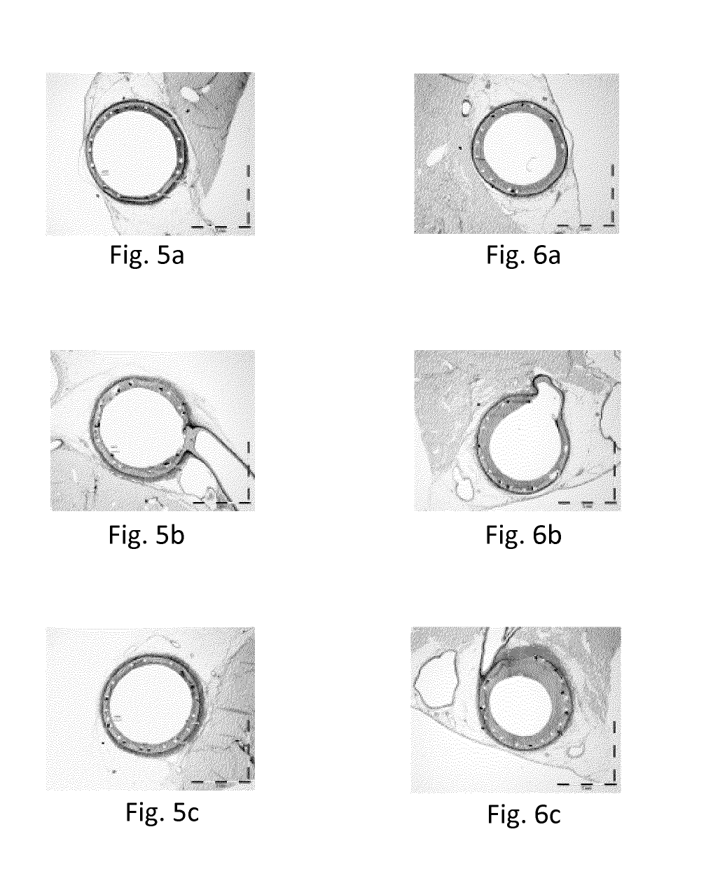

FIG. 5a, b, c: representation of the histomorphometric analysis of a stent with uniform cleanliness according to the invention.

FIG. 6a, b, c: representation of a histomorphometric analysis of the same stent type, as in FIGS. 5a, b, c with usual cleanliness.

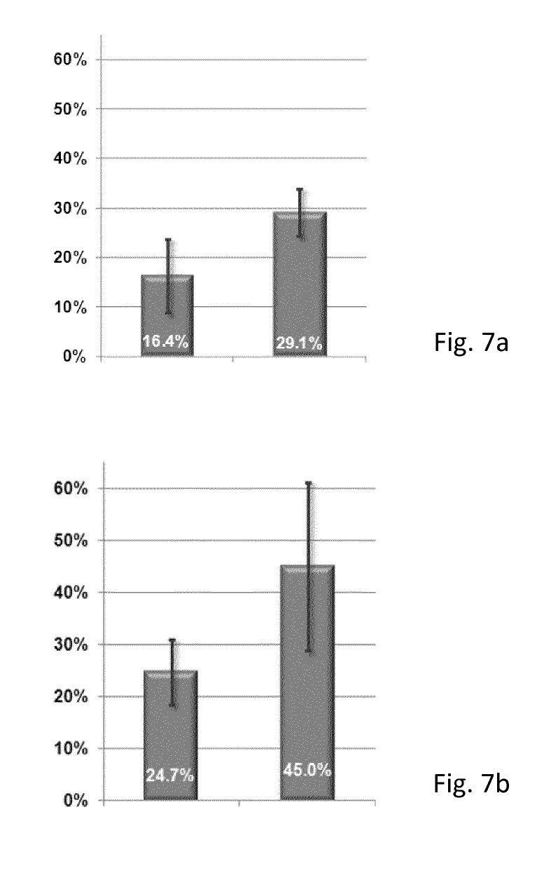

FIG. 7a: graphic representation of an in-vivo X-ray analysis (angiography) of a first stent according to the invention.

FIG. 7b: graphic representation of an in-vivo X-ray analysis (Angiography) of a second stent according to the invention.

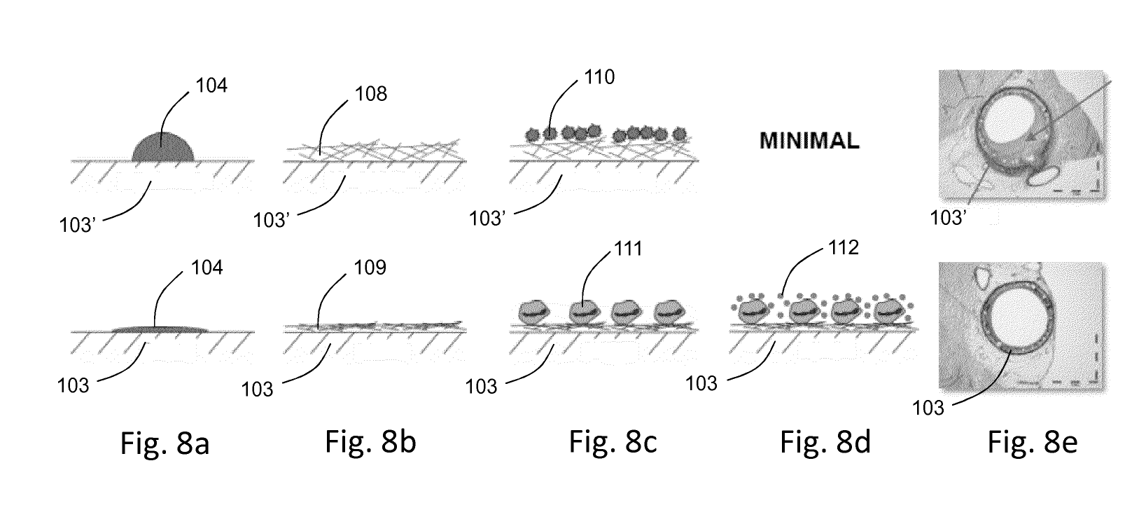

FIGS. 8a to 8e: schematic course of the growing in of a conventional bare metal stent (above) and a highly pure metal stent according to the invention (below).

In the following the preparation of an implant for implantation according to the invention is shown using the example of a stent. In general in the case of a stent with a balloon catheter as transport device there is the following procedure:

1. Stent is inserted in the tubular sheath.

2. A defined medium is made to flow around the stent

3. Balloon catheter is put in, and likewise has medium flowing around it

4. After a while, when medium flows around stent and balloon and thus no particles are located anymore on their surfaces, the balloon can be pushed into the stent lumen.

5. In an example, the balloon is opened somewhat (inflated), so that the stent remains on the balloon by means of frictional adhesion

6. Balloon catheter with stent is then inserted into a crimp unit inside the sheath and is reduced in diameter—stent and balloon.

7. Balloon catheter with crimped or installed stent is pushed into the packaging; the packaging is sealed tightly.

Shown in the FIGS. 1a to 1d is a first variant for carrying out a method according to the present invention, which is intended for the preparation of a balloon-expanding stent. As can be learned from FIG. 1a, the arrangement comprises a processing unit in the form of a crimp unit 1. Shown schematically are two crimp elements 1a and 1b of the crimp unit. The unit of course has more than two crimp elements. Furthermore the arrangement has a sheath 2 extending through the crimp unit 1 and beyond along the processing axis of the crimp unit, and which has a front opening 3 and a rear opening 4. The sheath 2 is provided as elongated tube, and is flexible as well as reducible in its diameter, or respectively can be folded. On the front opening 3 a flow unit 5 is attached. Provided in the region of the rear opening 4 is a cleaning unit 6, such as e.g. an ultrasound element. The flow unit 5 generates a flow of a defined medium in direction of the arrow through the sheath. Disposed inside the sheath 2 is an implant in the form of a stent 7 and a transport device in the form of a dilation catheter 8 with a dilation balloon 9. The stent shown in an expanded state before it is compressed on the balloon. Both the stent 7 as well as also the catheter 8 with the balloon 9 have the cleaning unit running through, in which they were cleaned, and are now flowed around by the flow of the defined medium from the flow unit 5 while they are transported in transport direction in direction of the flow unit inside the sheath. Stent and transport device are thus moved against the flow direction inside the arrangement, corresponding to a retrograde movement direction of the flow with respect to the stent and the transport device. A filter can be connected to the flow unit in order also to hold back contamination particles from the flow unit and prevent furthermore contamination.

As shown in FIG. 1b, in a next method step, the balloon 9 of the catheters 8 is disposed inside the stent 7 and both are stored in the crimp unit 1 between the crimp elements 1a and 1b. Meanwhile the medium continues to flow around the stent 7 and the transport device, so that no impurities from the shaft of the transport device, e.g. the balloon, remain between stent and balloon and are not able to be deposited on the stent. Thereby, owing to the retrograde flow, no contamination particles from the shaft of the transport device or the like remain between the transport device, e.g. the balloon, and the stent. The flow takes place thereby along the longitudinal axis of the crimp unit 1 and thereby enters the unit on one side and comes out of the unit again on the opposite side thereof. A modification of the components of the crimp device is thus not necessary for implementation of the sheath 2 and for conveyance of the flow.

In the next method step according FIG. 1c, the crimp elements 1a and 1 b of the crimp unit 1 compress the stent 7, so that it is reduced in its diameter and is disposed on the balloon 9. The sheath 2 thereby remains between the outer surface of the stent 7 and the crimp elements of the crimp unit 1. The defined medium continues to flow around the stent 7 also during the compression until its surfaces rest on the balloon 9. A minimal flow can e.g. flow through between the crosspieces of the stent or the flow is interrupted for a short time with a closing of the passage of the sheath during the compression. A deposit of particles between balloon 9 and stent 7 is prevented by the medium flowing by, which carries particles with it, which have e.g. possibly become detached from the catheter shaft during the crimping.

Next, as shown in FIG. 1d, a stent 7, compressed on the balloon 9, is led, inside the sheath 2, together with the catheter 8, out of the crimp unit 1. From there it can be put directly into a packaging. The sheath can thereby protrude at least partially into the packaging or can come out into an opening of the packaging.

The stent is moved, in this arrangement, against the flow direction P, so that the stent and the front (distal) part of the transport device always have fresh medium flowing around it.

Shown in the FIGS. 2a to 2c is a second variant for carrying out a method according to the present invention, intended for preparation of a self-expanding stents. The arrangement for preparation of the stent is comparable to that of FIGS. 1a to 1d. Foreseen once again is a crimp unit 1 with crimp elements 1a and 1 b, a sheath 2, which passes through the crimp unit 1, and a flow in direction of the arrow P. Flow unit and cleaning unit are not shown in favor of better clarity. Provided in the sheath are a stent 7 and a transport device. The transport device, or respectively the tube catheter, comprises an inner tube 10, a support tube 11 and an outer tube 12. Provided on the inner tube 10 is a proximal X-ray marker 13a, which serves in conventional way for checking the position of the stent in the body lumen. Disposed at the end of the inner tube 10 is a catheter tip 14, which defines the furthermost point during insertion of the stent in a body lumen and likewise has a (distal) X-ray marker 13b. The inner tube 10 is disposed over a guide wire and forms a passage to the tip 14. The support tube 11 is contiguous to the proximal X-ray marker 13a. The outer tube 12 is disposed above the support tube 11 and the inner tube 10.

In FIG. 2a the stent and the transport device are already cleaned and put into the sheath 2. The stent 7 is in an expanded state by means of the inner hose 10 between the two x-ray markers 13a and 13b positioned. The stent circumference is still larger in this state than the inner circumference of the outer tube 12. The stent 7 and the ends of outer tube 12 and support tube 11 are stored in front of the crimp elements 1a and 1b of the crimp unit.

As can be seen in FIG. 2b, the stent 7 is compressed by means of the crimp unit 1. In the compressed state the stent lies between marker 13a and 13b. The outer diameter of the compressed stent 7 corresponds now substantially to the diameter of the support tube 11. The stent is held by the crimp elements 1a and 1 b in compressed state, while it is cooled down below its austenite formation temperature in order to maintain the compressed state. For cooling of the stent, e.g. the temperature of the defined medium can be cooled or another medium with correspondingly lower temperature can be introduced in the sheath 2 which is added to the flow of the defined medium or replaces this. Alternatively a cooling medium can be provided outside the sheath.

Below the austenite formation temperature the stent remains in compressed state and the crimp elements 1a and 1b can be opened without the stent expanding, as is shown in FIG. 2c. The diameter of the stent is now still smaller than the inner diameter of the outer tube 12, so that this can be pushed over the stent and thus an undesired expanding of the stent prevented. Then the stent can be transferred into a packaging.

Before and during the compression of the stent and with further transfer, the flow of the defined medium flows around the stent and also the transport device. A contamination of the stent and deposits between stent and outer tube and/or inner tube are thus prevented.

Shown in FIG. 3 is a further arrangement for preparation of a stent for implantation according to the present invention. The arrangement shows a crimp unit 1, a sheath 2, a flow unit 5, a cleaning unit 6 and a packaging 15. Inside the sheath 2, a transport device 8 and a stent 7 are stored in compressed state, and can be moved inside the sheath 2. The sheath 2 extends from the or respectively through the cleaning unit 6 and through the crimp unit 1 to a first opening 16 of the packaging 15, and is connected to this opening. The flow unit 5 is attached at a second opening 17, which is opposite the first opening 16. The flow of the defined medium in direction of arrow P thus flows from the flow unit 5 through packaging 15 into the sheath 2, and flows around the stent 7 and the transport device 8 within the sheath 2. The stent 7 and the transport device 8 can be inserted within this flow into the packaging 15. Then the first and the second opening 16 and 17 of the packaging are closed, so that stent and transport device is stored inside the protective medium in the packaging. It is thereby advantageous, for the time being, to close the first opening 16 of the packaging, at which the sheath is provided, so that through the second opening 17 fresh medium is able to flow into the packaging and this remains until the second opening 17 is closed and thus is decoupled from the flow unit. For closing of the opening 16 and 17, gaskets 19, e.g. in the form of O-rings are used, which, owing to the closure (lid) 18 are compressed in such a way that the opening is sealed off around the transport unit on both sides of the stent 7. The lid 18 can compress the gaskets 19 e.g. through a rotation mechanism with a rotation relative to the packaging housing. The packaging is made of hard material. Thus it serves for protection of the stent and of the transport device from mechanical effects, and keeps these in their inner space in a defined medium, for example in a partially fluid and partially gaseous medium. A contamination of the stent during the preparation for implantation is thus prevented. Together with its transport device in the packaging, the stent is now ready for implantation.

Shown in FIG. 4 is an alternative packaging 20, in which the sheath 2 extends through the packaging 20. The sheath runs through a first opening 21 and a second opening 22, opposite thereto, of the packaging 20. A flow in direction P flows around the stent 7 and the transport device 8 inside the sheath 2 and the packaging 20. Provided at the openings 21 and 22 are closures 23 with seals 24, which close the packaging and the sheath tightly, for example with a rotation mechanism, as described in FIG. 3. The packaging 20 is made of hard material, and serves as protective sleeve for stent and transport device. The sheath 2 encloses the stent and the transport device and keeps this in a defined medium.

Shown in the FIGS. 5 to 7 are the results of animal studies which were obtained with insertion of stents in coronary arteries in pigs. In FIG. 7, the most minimal arterial diameter in the region of a stent was determined immediately after the insertion of a stent in an artery and thirty days after the insertion and growing in. After the insertion of the stent, the diameter of the stent decreases owing to the growing in of cells in the region of the stent. The arterial diameter is thereby angiographically captured, i.e. by means of X-ray analysis. The study comprises a measurement series of eight comparative trials.

Shown in the FIGS. 5a, 5b and 5c as well as 6a, 6b and 6c is the cross section of a coronary blood vessel of a pig, after it was examined after insertion of a stent by Medtronic, as is common for histological examinations. The original vessel rim is thereby identified, and then the cross-sectional surface of the grown-together cells calculated, from which a perceptual growth rate results, i.e. the endothelialization or respectively stenosis rate.

In the FIGS. 5a, 5b and 5c, a stent according to the invention is used with a uniform and highly pure surface, as can be verified e.g. according to the above-mentioned parallel application. Used was a commercially available stent of the manufacturer Medtronic of the size 3×18 mm and prepared by the method according to the invention. FIG. 5a shows the grown-in stent in a proximal region of the blood flow, FIG. 5b in a middle region with a bifurcation, and FIG. 5c in a distal region of the blood flow. As can be seen, only a minimal reduction of the diameter of the artery exists owing to ingrowth. Moreover there is a regular ingrowth of the stent.

Used in the FIGS. 6a, 6b and 6c is a conventionally prepared stent, which has no uniform pure surface, as was likewise able to be verified by the method according to the above-mentioned application. Both in the proximal region (FIG. 6a), in the middle region (FIG. 6b) as well as in the distal region (FIG. 6c), the diameter was significantly narrower than with the stent from the FIGS. 5a, 5b and 5c. In particular in the distal region, a strong restenosis can be shown.

As can be learned, through a uniform and pure stent surface, the risks can be significantly reduced in the treatment of arterial diseases. Moreover with the method and the device according to the invention, it is possible to determine reliably and without any great effort and expense, the surface characteristics of stents corresponding to a manufacturing method or a certain preparatory process. Thus stents which do not show any good surface characteristics can be excluded from implantation.

Used in FIG. 7a was a stent of Medtronic (3×18 mm) with a spiral-shaped grid structure, which was ready for implantation in a body lumen. The left bar shows the behavior of such a stent in the body lumen according to a preparation according to the invention with a uniform surface wetting behavior, and the right bar such a stent immediately out of the packaging without further treatment steps, as it is conventionally used. It is clear that the stent according to the invention with a uniform surface shows an improved ingrowth behavior, in contrast to a conventionally prepared stent. An undesired restenosis can, in this example, be reduced with a stent according to the invention from 29.1% to 16.4%, that is by about 44%, this being statistically significant (p=0.009).

Used in FIG. 7b was a stent of Abbott of the size 3×18 mm, which had a regular symmetrical, i.e. a non-helical, grid structure and was ready for implantation in a body lumen. The left beam shows once again the behavior of such a stent in the body lumen after preparation according to the invention and the right beam of such a stent immediately out of the packaging without further treatment steps, as conventionally used. Also the stent with this grid structure shows, after a preparation according to the invention, a clearly improved behavior during ingrowth in the artery with a significantly reduced narrowing of the diameter from 45% to 24.7%, i.e. by about 45%, this being statistically significant (p=0.024).

It clearly follows from these results that the use of a stent according to the invention with a uniform, or respectively periodically regular surface wetting behavior shows a clearly improved growing-in behavior and less restenosis, compared with conventionally available stents. According to the invention, therefore, bare metal stents provided with a uniform or respectively periodically regular surface wetting behavior, i.e. highly pure stents, are to be provided for the implantation in body lumina.

In the FIGS. 8a to 8e there is a schematic representation of the individual steps during growing in of a stent, as was able to be identified during trials of the applicant. Shown are the individual steps with a conventional bare metal stent 103′ (above) and a highly pure bare metal stent 103, in particular with a stent having a uniform surface wetting behavior over its entire length, or respectively a periodically regular surface wetting behavior. The highly pure stent was obtained through the behavior and the arrangement according to the parallel application CH 00049/12 of the applicant. In the example according to the FIGS. 8a to 8e, a stent is considered with a highly hydrophilic surface.

Shown in FIG. 8a is the wetting behavior of a water drop 107 on the stent surface. On the conventional stent 103′ (FIG. 8a, above) there is a large angle of contact whereas with the highly pure stent 103 (FIG. 8a, below) there arises only very minimal contact angle owing to the hydrophilic surface characteristics. In FIG. 8b the stent is placed at the site of the implantation and the considered surface is exposed with respect to blood. With the conventional stent 103′ (FIG. 8b, above) there takes place first a deposit of proteins, which prevent both the adhesion as well as also the functionality of neutrophiles (FGN, A2M, ApoA), so-called neutrophil inhibitors 108. With the highly pure stent 103 (FIG. 8b, below), the neutrophil inhibitors 108 (FGN, A2m and ApoA) are greatly reduced and at the same time proteins are deposited, which prevent the adhesion of thrombocytes (HMWK), as well as proteins, which promote the adhesion of neutrophils on the stent surface (PGN), so-called neutrophil promoters 109. Correspondingly then, with the conventional stent 103′ (FIG. 7c, above) mainly thrombocytes 110 are settled on the neutrophil inhibitors 108, which is basically undesired. With the highly pure stent 103 (FIG. 8c, below) on the other hand, neutrophilic cells 111 from the blood of the patient are settled on the neutrophilic promoters 109, whereas thrombocytes are rejected. The activated neutrophilic cells 111 separate the protein cathelicidin (LL37) 112 on the stent surface, cf. FIG. 8d below. The process of the growing in can thereby be supported in a positive way without the use of coating or the delivery of drugs. With the conventional stent 103′ cathelicidin was found only in very minimal quantity. The studies have shown that the highly pure stent 103 accumulates two to three times more cathelicidin than the conventional stent. The highly pure stent 103 with a regular surface wetting behavior shows an ingrowth behavior comparable with that from the FIGS. 5a to 5c (see FIG. 8e, below). The conventional stent 103′ shows however a resumption of growing-in according to the FIGS. 6a to 6c, i.e. another narrowing of the passage (see FIG. 8e, above). In summary, it can be concluded that the highly pure surface of the stent 103 supports and promotes those bioactive processes that lead to a healthy and desired growing in of the stent 103. Undesired processes, on the other hand, are inhibited or stopped.

### LIST OF REFERENCE NUMERALS

1 crimp unit
2 sheath
3 front opening
4 rear opening
5 flow/L unit
6 cleaning unit
7 implant, stent
8 transport device
9 balloon
10 inner tube
11 support tube
12 outer tube
13a, 13b marker
14 catheter tip
15 packaging
16 first opening
17 second opening
18 closure
19 gasket
20 packaging
21 first opening
22 second opening
23 closure
24 gasket
103, 103′ stent
107 water drop
108 neutrophilic inhibitors
109 neutrophilic promoters
110 thrombocytes
111 neutrophilic cells
112 cathelicidin
P flow direction

1. Arrangement for preparation of an implant for implantation in a body lumen comprising:
at least a processing unit for preparation of the implant,
a flexible sheath with a front opening and a rear opening, which is disposed at least partially on or in the processing unit, the implant being inside the sheath being able to be stored between these openings, and
at least one flow unit for generation of a flow of a defined medium between the front opening and the rear opening through the sheath, which is connected at least at an opening of the sheath, the flow flowing through the implant inside the sheath.

2. Arrangement according to claim 1, wherein the sheath being designed tubular and/or flexible.

3. Arrangement according to claim 1, wherein the implant being movably arranged inside the sheath.

4. Arrangement according to claim 1, wherein, in or on the sheath, holding means for positioning of the implant inside the sheath being provided.

5. Arrangement according to claim 1, wherein the sheath has a wall thickness of 0.01 mm to 0.09 mm, in particular from 0.02 mm to 0.06 mm.

6. Arrangement according to claim 1, wherein the sheath is composed of PTFE or PFA.

7. Arrangement according to claim 1, wherein the sheath extending through the processing unit, in particular a packaging unit, and emerging therefrom at opposite sides of the processing unit.

8. Arrangement according to claim 1, wherein a plurality of processing units are provided in a series and the sheath extends through the plurality of processing units.

9. Arrangement according to claim 1, wherein the sheath is able to be removed from the processing unit and/or is movable therein.

10. Arrangement according to claim 1, wherein the processing unit comprises a cleaning unit, a unit for compression of the implant (crimp unit) or a packaging unit, or respectively a packaging.

11. Arrangement according to claim 1, wherein disposed between the flow unit and the sheath is a packaging.

12. Arrangement according to claim 1, wherein, for insertion of the implant into a body lumen, a transport device is provided, together with the implant in the sheath between its openings.

13. Arrangement according to claim 1, wherein a packaging unit is disposed in flow direction at the front after a flow unit, and is connected by an opening to the flow unit and by an opposite opening to an opening of the sheath.

14. Arrangement according to claim 1, wherein provided as medium is a physiological sodium chloride solution or WFI water.

15. Method for preparation of an implant for implantation in a body lumen, in which the implant is inserted inside a flexible sheath of a processing unit, wherein the implant is stored between a front opening and a rear opening of the sheath and a flow of a defined mediums is conducted through the sheath between the front opening and the rear opening which flows around the implant inside the sheath at least partially, and the processing unit acts upon the implant from outside the sheath.

16. Method according to claim 15, wherein the flow flows around the implant while the implant is cleaned, prepared, is fed to a packaging and/or is transferred between units.

17. Method according to claim 15, wherein the implant remains inside the sheath during a transfer from a cleaning unit, for cleaning of the implant, to one or more processing units and/or to a packaging.

18. Method according to claim 15, wherein the implant, together with the sheath, is brought into a processing unit, in particular a crimp unit, and is removed therefrom.

19. Method according to claim 15, wherein a transport unit for insertion of the implant in a body lumen is stored together with the implant in the sheath.

20. Method according to claim 15, wherein the flow of the defined medium takes place in a retrograde way with respect to a transport direction of an implant within an arrangement consisting of at least a cleaning unit as well as a processing unit and/or a packaging.

21. Method according to claim 15, wherein the temperature of the flow is changed during the preparation of the stent.

22. Method according to claim 15, wherein at least one additional substance is added to the flow.

23. Method according to claim 15, wherein the strength of the flow is adjustable in a variable way.

24. Method according to claim 15, wherein a packaging unit is provided, in which the implant inside the sheath is packaged together therewith in a packaging.

25. Method according to claim 15, wherein a packaging unit is disposed in flow direction in the front after a flow unit and a defined medium flows around it, the sheath being connected to the packaging unit.

26. Implant in the form of a stent, which is prepared according a method of claim 15 for implantation.

27. Implant according to claim 26, wherein the total surface of the stent has a consistent cleanliness with fewer particles per square meter than corresponds to a cleanroom class 1 of the ISO (International Standard Organisation).

28. Implant according to claim 26, wherein the stent has fewer particles of a size under 0.05 micrometer than corresponds to cleanroom class 1 of the ISO (International Standard Organisation).

29. Packaging of an implant for implantation in a body lumen, which has a tubular flexible sheath with a front opening and a rear opening, between which the implant is stored, the sheath being provided inside the packaging and the openings of the sheath are sealed by the packaging or parts of the packaging.

30. Packaging according to claim 29, wherein at least at one of the openings of the sheath a connection is provided for a flow unit for generation of a flow of a defined medium through the sheath.

31. Packaging according to claim 29, wherein the packaging has a housing of hard metal.
